(19) United States
(12) Patent Application Publication
(10) Pub. No.: US 2015/0164331 A1
(43) Pub. Date: Jun. 18, 2015

(54) INTEGRATED SYSTEM ARCHITECTURES

(71) Applicant: VOLCANO CORPORATION, San Diego, CA (US)

(72) Inventors: Vincent Burgess, San Diego, CA (US); Timothy K. Glynn, San Marcos, CA (US)

(73) Assignee: VOLCANO CORPORATION, San Diego, CA (US)

(21) Appl. No.: 14/342,197

(22) PCT Filed: Aug. 30, 2012
(86) PCT No.: PCT/US2012/053171

§ 371 Date: Nov. 13, 2014

Related U.S. Application Data
(60) Provisional application No. 61/529,752, filed on Aug. 31, 2011.

(51) Int. Cl. A61B 5/00; A61N 7/02; A61B 5/01; A61B 5/055; A61B 8/12

(52) U.S. Cl. CPC A61B 5/0046; A61B 5/055; A61B 5/0066; A61B 8/12; A61B 5/0075; A61B 5/015; A61B 5/0095; A61B 5/0013; A61N 7/02

(57) ABSTRACT

The invention generally relates to imaging systems and more particularly to integrated architectures. In certain embodiments, the invention provides an integrated system including a work station and a patient area, in which the work station is remote from the patient area and the work station is operably associated with the patient area.

### RELATED APPLICATION

The present application claims the benefit of and priority to U.S. provisional application Ser. No. 61/529,752, filed Aug. 31, 2011, the content of which is incorporated by reference herein in its entirety.

### FIELD OF THE INVENTION

The invention generally relates to imaging systems and more particularly to integrated architectures for imaging systems.

### BACKGROUND

Intravascular imaging systems generally employ an architecture consisting of CPU components on a roll-around cart with the sample path of an interferometer extending (≈3 m) to the patient via a non-user-disconnectable Patient Interface Module (PIM) or a Patient Interface Unit (PIU) or a DOC. The short PIM cable forces the system to be located physically near the patient to avoid problems associated with long separation distance (i.e. optical dispersion and z-offset perturbation) and a permanently connected PIM cable avoids problems with connector damage/debris (i.e. insertion loss), which is difficult to avoid in the catheter lab environment when users are not trained fiber optic technicians. A problem with mobile intravascular imaging systems is that they are large and difficult to maneuver within the confines of a busy hospital, particularly in a catheterization laboratory.

### SUMMARY OF THE INVENTION

The invention generally relates to system architectures for integrated systems. The invention recognizes that valuable space can be saved in a catheterization laboratory by locating certain components of an imaging system in an area remote from other components of the system and then operability associating the components with each other. In this manner, architectures of the invention minimize the amount of hardware that must be located by the patient and provide valuable space in the vicinity of the patient.

The integrated systems of the invention generally include a control room and/or a work station that is remote from the patient table and a patient area where some portion of the integrated system resides in close proximity to the patient table allowing a user to connect an imaging device via a bedside interface. The control room and/or work station is operably associated with the patient area and the control room or work station is remote from the patient area. In most set-ups, the control room will be a substantial physical distance from the patient area, e.g., about 5 m or greater.

### BRIEF DESCRIPTION OF THE DRAWINGS

In the accompanying figures, like elements are identified by like reference numerals among the several preferred embodiments of the present invention.

FIG. 1A is a schematic diagram of an integrated system; FIG. 1B is a schematic diagram of an extended interferometer sample path; and FIG. 1C is a schematic diagram of an extended source path and detector system.

FIG. 2A is a schematic diagram of an extended digitizer-CPU system; FIG. 2B is a schematic diagram of an extended digitizer-CPU mobile system; and FIG. 2C is an extended digitizer-CPU laptop system.

FIG. 3A is a schematic diagram of a dual light path and digital PIM cable system; FIG. 3B is a schematic diagram of a dual light path and analog PIM cable system; FIG. 3C is a schematic diagram of a PIM integrated interferometer system; and FIG. 3D is a schematic diagram of a PIM integrated interferometer system.

FIG. 4A is a schematic diagram of a distributed interferometer system; FIG. 4B is a schematic diagram of another distributed interferometer system; FIG. 4C is a schematic diagram of another distributed interferometer system; FIG. 4D is a schematic diagram of another distributed interferometer system; FIG. 4E is a schematic diagram of another distributed interferometer system; and FIG. 4F is a schematic diagram of another distributed interferometer system.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting the scope of the invention being defined by the appended claims and equivalents thereof.

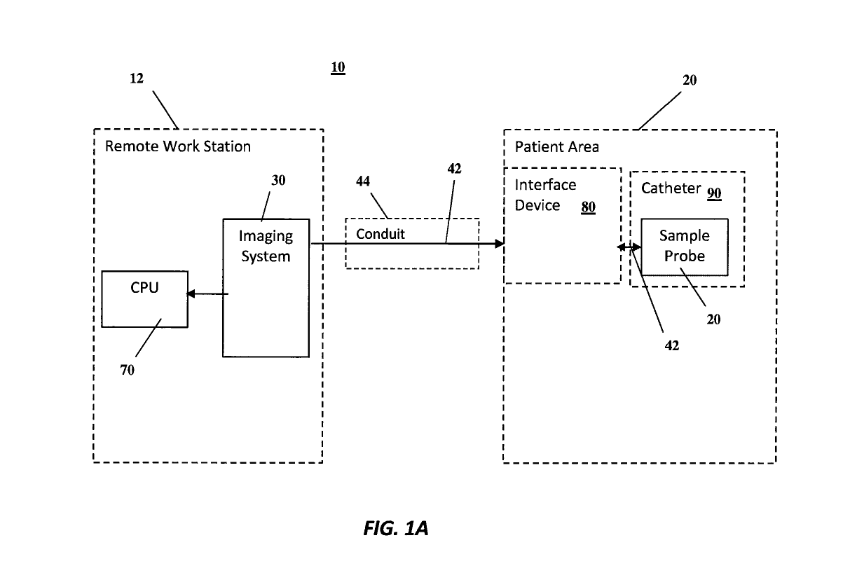

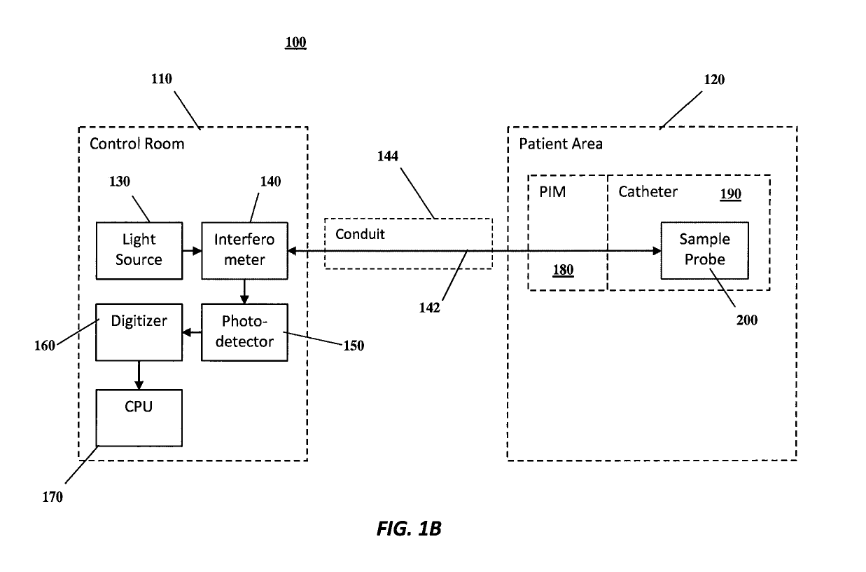

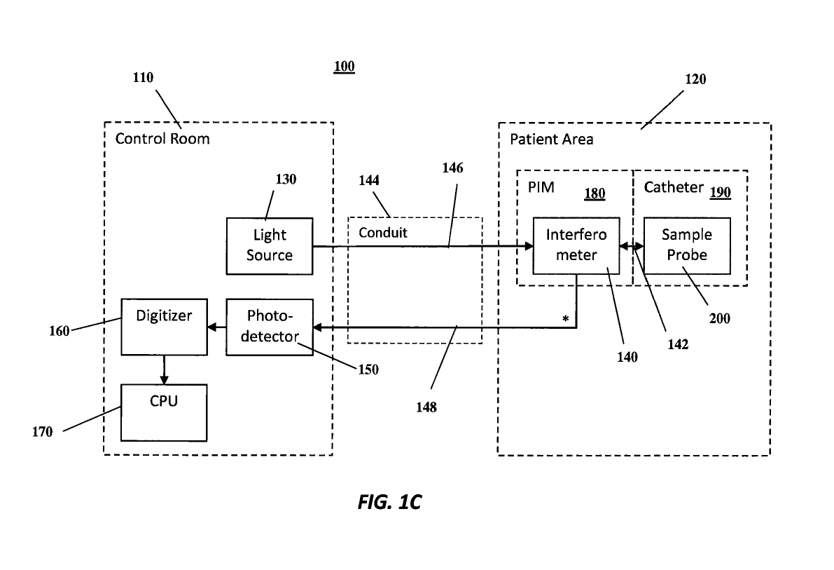

Generally speaking, a variety of architecture concepts is based on an integrated system comprising a central processing unit (CPU) that is located a substantial physical distance from a sample, as shown in FIGS. 1-4. In one embodiment, the sample is a patient's vessel located within a patient area; alternatively, the sample is in any surgery suite, operation room, patient care area, operation site, and the like. The integrated systems are designed to locate the sample a substantial physical distance away from an imaging system's central processing/display/archival unit, as is necessary for cardiac catheterization lab and other procedural patient room integration including inpatient and outpatient surgical suites that are appropriate settings for the use of imaging devices (e.g. control room or remote work station separated from patient table by multiple meters). As described herein, a substantial physical distance is greater than at least 5 m, alternatively, greater than at least 10 m, alternatively, between at least 1 and 1000 m. In one embodiment, a substantial physical distance may be inside of a control room or other remote location away from the sample.

The present architectures are described herein as the imaging systems relate to Optical Coherence Tomography (OCT) systems; however, the integrated systems may also be applied to other imaging systems, including by way of example and not limitation, such as spectroscopic devices, (including fluorescence, absorption, scattering, and Raman spectroscopies), intravascular ultrasound (IVUS), Forward-Looking IVUS (FLIVUS), high intensity focused ultrasound (HIFU), radiofrequency, thermal imaging or thermography, optical light-based imaging, magnetic resonance, radiography, nuclear imaging, photoacoustic imaging, electrical impedance tomography, elastography, pressure sensing wires, intracardiac echocardiography (ICE), forward looking ICE and orthopedic, spinal imaging and neurological imaging, image guided therapeutic devices or therapeutic delivery devices, diagnostic delivery devices, and the like.

In one embodiment, as shown in FIG. 1A, the integrated system 10 comprises a remote work station or control room 12 and a patient area 20, whereby the remote work station 12 is operably associated with the patient area 20 at a substantial physical distance. The remote work station 12 includes an imaging system 30 and a CPU component 70. The patient area 20 includes an interface device 80 and a catheter 90 and a sample probe 20 operably associated with the catheter 90 by way of a connection path 42. The imaging system 30 is operably associated with the patient area 20 by way of the interface device 80 through a conduit 44. The conduit 44 may include an optical fiber, electrical or wireless communication channel 42 to communicate the imaging system 30 with the interface device 80. The CPU 70 is operably associated with the imaging system 30 to enable the separation of CPU components from the sample by a substantial physical distance, as shown in FIG. 1A. In another embodiment, the integrated systems enable installation of CPU components and cables in a permanent fashion through the conduit but preserve portability and modularity of the patient interface device and catheter components, as shown in FIGS. 1-4. In another embodiment, the integrated systems enable multiple instances of the patient interface components located in various locations to interface with a single set of CPU components. The CPU components may be connected with the sample probe by way of wires, cables, optical fibers, wireless communications, and the like. Communication between any proximal and distal ends of any part of the device, system, or apparatus may be by any communication devices, such as wires, optics, wireless, RF, and the like.

In another embodiment, the integrated systems comprise an electronic subsystem that generates image data in some remote location and converts the data to digital form, as shown in FIGS. 1-4. In one embodiment, a digitizer converts the image data to digital form. This digital data is transmitted across a network and received on the opposite end of the network with another subsystem which performs other tasks (archival, analysis, display) on the data.

Generally, in an optical system for transmitting digital information, the component used to convert electrical data stream to/from the optical data stream is an optical transceiver, which is a component for high-speed optical networking. Command and control signals can also be transmitted on the network, in addition to the image data. The integrated system may include a plurality of optical transceivers and optical fibers and a plurality of wires or wireless channels can be used. High-bandwidth and long-distance image/data transmission from a remote system to a host computer uses a digital network comprising a physical layer. In one embodiment, the network's physical layer comprises an optical communication (e.g. fiber optic), an electrical communication (e.g. copper wire or coax cable for CP/IP, UDP, Firewire, USB 2, SCSI, SATA, eSATA, PCI, PCI-Express, IDE, etc.), or wireless communication (e.g. WiFi, Radiofrequency, Bluetooth, mobile communication, and the like). The digital data transfer across the network can be in serial or parallel transfer.

The term “Network” is not limited to specific consumer/commercial embodiments (such as Ethernet, USB, or Firewire), but includes any system of at least two individual members (e.g. system and host computer) that are interconnected by a communications channel in order to transmit information (e.g. image data). Image/data compression reducing transfer bandwidth can include loss compression or lossless compression. In one embodiment, the remote CPU performs decompression on a compressed incoming data stream.

Additionally, embodiments disclosed herein solve bandwidth limitations of networks by first performing image compression (e.g. JPEG or other) within the remote system before transmitting image data to the host over the network. The image compression reduces the bandwidth necessary to transmit the image data over a substantial physical distance. A remote, network-connectable system includes system front-end components (e.g. light source, interferometer, digitizer, etc) that can be kept in close proximity to the sample being imaged, versus extending the interferometer (long sample arm fiber) or source/detection path fiber optics. When the front-end system is located remotely and the transfer of information to a host computer is via a digital network transfer, a wider variety of system installation options is enabled.

Generally speaking, the method for integrating the systems with a catheter lab or other patient procedural area comprise locating the physician/patient interface components and disposables in proximity to sample; and locating the non-portable hardware a substantial physical distance away. In one embodiment, the components and disposables include the controllers, PIM, and imaging catheter. In one embodiment, the non-portable hardware includes the CPU components, power supplies, display monitors, and archival system. Generally speaking, the CPU components include power supplies, display monitors, archival system and the like, may be generally referred to as the “CPU components”, and are further explained below.

The method for integrating the system further comprises connecting a patient/physician interface components with CPU components. In one embodiment, the connecting patient/physician interface includes permanently installed cables (electrical or optical) or wireless transmission. In another embodiment, the installed cables may be through a conduit, which may be a floor trench, a ceiling conduit, air for wireless transmission, and the like.

The method for integrating the systems further comprises disconnecting the patient interface components from the permanently installed components when the patient interface components are not in use, need repair, substitution, or updating. This embodiment allows for modularity, portability, serviceability, and the like of the integrated systems.

The method further comprises separating the system from its host computer and connecting with a network cable at a substantial physical distance (rather than direct host bus slot, i.e. PCI/e) to enable imaging system portability and ability to quickly interchange imaging systems and hosts (e.g. server, desktop PC, laptop PC, netbook, mobile device, etc.)

In one embodiment, the image information is transmitted from the sample to the CPU components in a manner that does not substantially reduce the quality of the image or data. Image quality reduction includes noise (e.g. electrical interference or bit errors on copper cables or wireless transmission, lossy compression), group delay dispersion (e.g. an effect in a fiber interferometer which reduces resolution and is hard to manage in long fiber cables), z-offset perturbation (mechanical or thermal changes in interferometer fiber path length), and optical insertion loss (optical transmission compromised by bent or broken fiber or dirty/damaged optical connectors). The integrated systems disclosed herein are able to fulfill these basic integration requirements to reduce noise, group delay dispersion, z-offset perturbation, and optical insertion loss.

The integrated system may be used in other medical sub-specialties outside of interventional cardiology in which an integrated OCT system is important, such as other surgical suites. The OCT applications outside of medicine could also use these integrated OCT systems for materials characterization for manufacturing, chemical identification, optical fiber architectures, and the like. Other embodiments include OCT, cardiac catheterization lab integration, OCT system architectures, Optical Frequency Domain Interferometry (OFDI), Swept-Source OCT (SS-OCT), and alternative imaging systems described above, and the like.

Generally speaking, a swept-source Fourier-domain intravascular OCT imaging system comprises: a light source and an optical interferometer. In one embodiment, the light source includes a tunable laser, a tunable-superluminescent diode (TSLED) or other tunable light source of photons. Alternatively, a light source for any other optical based imaging system may include a laser, superluminescent diode (SLD), or any other source of photons. In one embodiment, the optical interferometer includes a sample path and a reference path. A “path” may be physically co-located in the same spatial location or fiber (e.g. “common path”) and can consist of a number of interferometer layouts (Michelson, Mach Zehnder, etc). Paths in the interferometer may be physically distributed over long distances and supported by fiber-optic transmission. The optical interferometer includes at least one fiber splitter/coupler or other beam-splitting/combining element for the sample and reference paths.

The OCT interferometer can be operably coupled to a sample probe. In one embodiment, the sample probe comprises a rotational catheter for intravascular imaging. In other embodiments, the sample probe includes an endoscopic probe, forward-imaging probes, galvo-scanners, or other alternative lateral scanning mechanisms for a variety of applications. The sample probe necessarily has to be located in close proximity to the sample/patient and is operably associated with the sample path of the interferometer. An exemplary sample probe is disclosed in commonly assigned U.S. patent application Ser. No. 12/172,922, incorporated by reference herein.

Additionally, the OCT interferometer is operably coupled to a photodetector or photoreceiver. The photodetector may include multiple detectors when using balanced detection and/or polarization diverse detection, e.g. splitting the sample path into separate polarization states and using at least two detectors to detect the separated polarization states. The OCT interferometer is operably coupled to a digitizer, which converts continuous analog OCT signals into sampled digital OCT signals. Analog pre-filtering and amplification are employed between the photoreceiver and digitizer.

The OCT interferometer is operably coupled with a computer or CPU component, which performs processing, display, archival, user interface, etc. functions of the system. In one embodiment, the CPU component includes multiple pieces of computing hardware distributed in different locations and interconnected with digital communication links. The CPU component can include standard PCs (desktops, laptops, servers, etc), embedded processors (Digital Signal Processors “DSP” and programmable logic arrays “PLA” such as field-programmable gate array “FPGA”, etc.), graphic cards (Graphic Processing Units “GPU”), and other computing hardware/software. For an integrated imaging system, the primary computer elements are located a substantial physical distance away from the sample/patient, i.e. in the control room or remote work station. The computer can be of various types including a personal computer, a portable computer, a network computer, a control system in surgical system, a mainframe, or a remotely controlled server.

In one embodiment, the processes, systems, and methods illustrated above may be embodied in part or in whole in software that is running on a computing device or CPU components. The functionality provided for in the components and modules of the computing device may comprise one or more components and/or modules. For example, the computing device may comprise multiple central processing units (CPUs) and a mass storage device, such as may be implemented in an array of servers. Multiple CPU's and GPU's may be in a distributed fashion, as more fully described in commonly assigned U.S. patent application Ser. No. 11/868,334, incorporated by reference herein.

In general, the word “module,” as used herein, refers to logic embodied in hardware or firmware, or to a collection of software instructions, possibly having entry and exit points, written in a programming language, such as, for example, Java, C or C++, or the like. A software module may be compiled and linked into an executable program, installed in a dynamic link library, or may be written in an interpreted programming language such as, for example, BASIC, Perl, Lua, or Python. It will be appreciated that software modules may be callable from other modules or from themselves, and/or may be invoked in response to detected events or interrupts. Software instructions may be embedded in firmware, such as an EPROM. It will be further appreciated that hardware modules may be comprised of connected logic units, such as gates and flip-flops, and/or may be comprised of programmable units, such as programmable gate arrays or processors. The modules described herein are preferably implemented as software modules, but may be represented in hardware or firmware. Generally, the modules described herein refer to logical modules that may be combined with other modules or divided into sub-modules despite their physical organization or storage.

In one embodiment, the CPU components comprises a mainframe computer suitable for controlling and/or communicating with large databases, performing high volume transaction processing, and generating reports from large databases. The CPU may comprise a conventional microprocessor. The CPU components further comprise a memory, such as random access memory (“RAM”) for temporary storage of information and/or a read only memory (“ROM”) for permanent storage of information, and a mass storage device, such as a hard drive, diskette, or optical media storage device. Typically, the modules of the computing system are connected to the computer using a standards based bus system. In different embodiments, the standards based bus system could be Peripheral Component Interconnect (PCI), Microchannel, SCSI, Industrial Standard Architecture (ISA) and Extended ISA (EISA) architectures, for example.

The example computing system and CPU components comprises one or more commonly available input/output (I/O) devices and interfaces, such as a keyboard, mouse, touchpad, and printer. In one embodiment, the I/O devices and interfaces comprise one or more display devices, such as a monitor, that allows the visual presentation of data to a user. More particularly, a display device provides for the presentation of GUIs, application software data, and multimedia presentations, for example. The I/O devices and interfaces also provide a communications interface to various external devices. The computing system may also comprise one or more multimedia devices, such as speakers, video cards, graphics accelerators, and microphones, for example.

In an alternative embodiment, the OCT interferometer includes a Variable Delay Line (VDL) in the either sample or reference path. The VDL is used to compensate for small pathlength variations in the interferometer during system use. The integrated OCT system may also include a Patient Interface Module (PIM), which is used in intravascular OCT systems for interfacing a rotational catheter with rotation and translation drive motors. Alternatively, PIM's may be any interface module to couple an imaging system component to the catheter, sample, or sample probe. The PIM component as designated can consist of either a single physical box or multiple separate boxes (separated with cables, wireless connections, and the like). For example, one interface module has the light source, detectors, digitizer, reference arm in the PIM box and the motor and catheter interface in a separate PIM box. Alternatively, the interface module may be a longitudinal pullback device, such as the Volcano™ Revolution™ PIM, the Volcano™ R100, or the Volcano™ Trak Back II Catheter Pull-Back Device, for operation of a rotational catheter or other imaging catheter.

In an alternative embodiment, the OCT interferometer includes a Sample Clock Generator. Light sources with non-linear sweep profiles must be accompanied by a sample clock generator which effectively synchronizes the light source output to the digitizer via a separate clocking interferometer (e.g. “wavemeter”) and photodetector subsystem. Light sources with linear (in k-space) sweeps can use a digitizer's internal (on-board) sample clock generator. The sample clock generator scheme is an important component for SS-OCT Like other components, its location can be distributed physically over a significant distance and can share common elements with the OCT system (interferometer, detectors, digitizer, and the like). An exemplary clock generator is disclosed in commonly assigned U.S. patent application Ser. No. 12/172,980, incorporated by reference herein.

In another embodiment, the OCT interferometer may be a “fiber-based” SS-OCT system. The SS-OCT system generally comprises a Light Source and an Optical interferometer in communication with the light source by a source path. The SS-OCT system comprises a sample path operably associated with a scanning probe. The scanning probe is in communication with the rest of the interferometer via optical fiber in the sample path. The SS-OCT system comprises photodetectors in communication with the Optical interferometer through the detection path. The photodetectors are in communication with the digitizers via analog signal transmission over electrical wires, commonly including electronic analog amplification/filtering stages. The digitizers are in communication with the CPU via digital communication (electrical, digital optical, or wireless; parallel or serial data transmission; computer data bus) or analog. An exemplary SS-OCT system is described in U.S. patent application Ser. No. 12/172,980, and incorporated by reference herein.

In a “non-fiber-based” SS-OCT system, the fiber components can be replaced with bulk optical components (beam-splitters, lenses, minors, polarizers, etc) and the optical beams are transmitted through open space. Photodetector/Digitizer/Computer connectivity remains the same.

In a Spectral Domain (spectrometer-based) OCT system, the same components are used with a few modifications. The light source is no longer tunable, but is a broadband short-coherence length source. The photodetectors are replaced with a spectrometer and detector array and the digitizer is usually referred to as a frame grabber, although its function is basically the same. All other basic system components and interconnectivity are the same.

Other intravascular imaging systems follow the same architectural paradigm of physically containing all system elements (except for the sample path which extends to the sample via the PIM and catheter) together inside a cart or mobile console. The digitizer is usually contained within the computer and is connected via a high-speed internal data bus of the computer (e.g. PCI, PCIe). The photodetectors can be located on the same card as the digitizer, as can some embedded processing units. Many specific configurations of the basic elements are possible, but all maintain the same physical co-location in a mobile cart. The integrated system architectures disclosed herein enable a paradigm in which the primary system elements are not physically co-located in the same cart or mobile console.

In one embodiment, the integrated OCT system 100 is shown in FIG. 1B, which is an extended interferometer sample path. The integrated OCT system 100 comprises a control room or a remote work station 110 and a patient area 120, whereby the remote work station 110 is operably associated with the patient area 120. The control room 110 may be any general area or location that is a substantial physical distance from the patient area 120, such as the remote work station. The control room 110 comprises a light source 130 operably associated with an interferometer 140, a photodetector 150 operably associated with the interferometer 140, a digitizer 160 operably associated with the photodetector 150, and a CPU 170 operably associated with the digitizer 160. The patient area 120 comprises a PIM 180 operably associated with a catheter 190, and a sample probe 200 operably associated with the catheter 190. The interferometer 140 includes an extended sample path 142 that operably associates with the sample probe 200 to integrate the control room OCT system with the Patient Area and PIM 180. Generally speaking, the extended sample path 142 is provided within a conduit 144, whereby the conduit may be an optical fiber, an electrical coupling, and the like. The integrated OCT system 100 locates the OCT sample a substantial physical distance away from the OCT system's central processing/display/archival unit. The CPU 170 in the control room 110 is for the imaging and processing of the images obtained from the catheter 190 and sample probe 200.

In another embodiment or architecture, the integrated OCT system 100 is shown in FIG. 1C, which is an extended source path and detector system. The integrated OCT system 100 in this embodiment comprises the control room 110 and the patient area 120, whereby the control room 110 is operably associated with the patient area 120 at a substantial physical distance. The control room 110 comprises the light source 130, the photodetector 150 operably associated with the digitizer 160, and the CPU 170 operably associated with the digitizer 160. The patient area 120 comprises the PIM 180 which includes the interferometer 140 operably associated with the sample probe 200 by the sample path 142, whereby the catheter 190 includes the sample probe 200. The light source 130 is operably associated with the interferometer 140 at a substantial physical distance by a source path 146 through the conduit 144, and a detection path is operably associated with the interferometer and the photodetector 150 through the conduit 144. If a Michelson interferometer is employed then a shared source path 146 and detection path 148 are used. If a Mach-Zehnder interferometer is employed, then a separate detection path 148 from the path 146 may be used. The CPU 170 in the control room 110 is for the imaging and processing of the images obtained from the catheter 190 and sample probe 200.

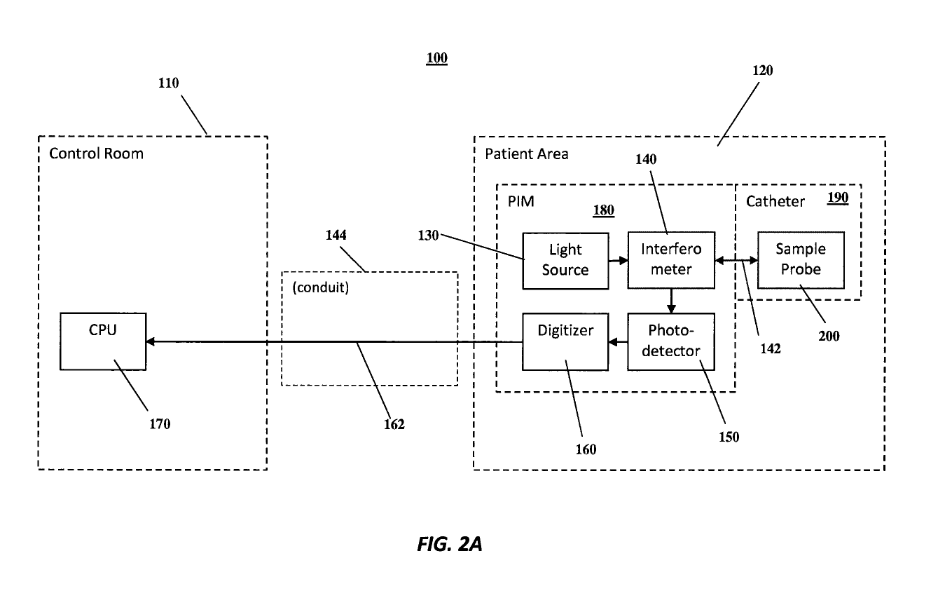

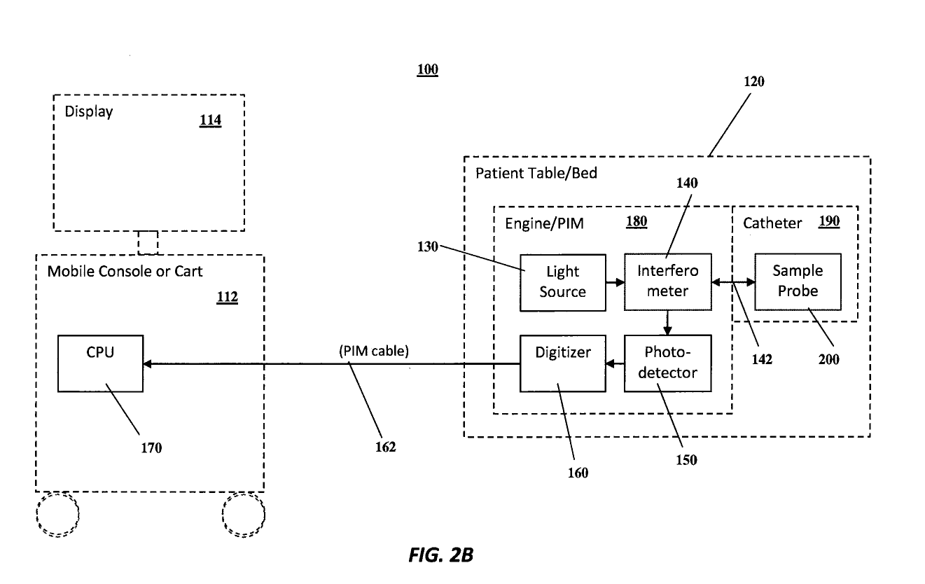

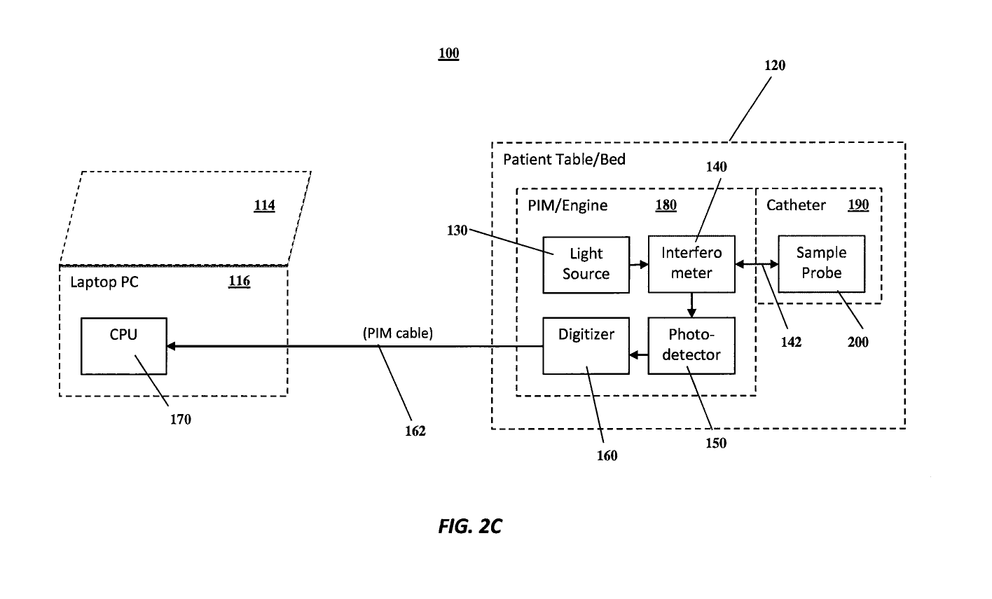

In another embodiment, the integrated OCT system 100 is shown in FIG. 2A, which is an extended digitizer-CPU system. The integrated OCT system 100 in this embodiment comprises the control room 110 and the patient area 120, whereby the control room 110 is operably associated with the patient area 120 at a substantial physical distance. The control room 110 comprises the CPU 170 and the patient area 120 comprises the PIM 180 and the catheter 190. The PIM 180 includes the light source 130, the interferometer 140, the photodetector 150, and the digitizer 160. The light source 130 is operably associated with the interferometer 140 within the PIM 180, while the interferometer 140 operably associated with the sample probe 200 by the sample path 142. With the interferometer 140 included in the PIM 180, the sample path 142 does not traverse a substantial physical distance, but is rather locally connected with the catheter 190. The interferometer 140 is operably associated with the photodetector 150 in the PIM 180, while the photodetector 150 is operably associated with the digitizer 160 within the PIM 180. The digitizer 160 is operably associated with the CPU 170 in the control room 170 by way of CPU path 162 that is operably associated with the conduit 144. The integrated OCT system 100 locates the OCT sample a substantial physical distance away from the OCT system's central processing/display/archival unit. The CPU 170 in the control room 110 is for the imaging and processing of the images obtained from the catheter 190 and sample probe 200.

In another embodiment, the integrated OCT system 100 is shown in FIG. 2B, which is an extended digitizer-CPU mobile system. The integrated OCT system 100 in this embodiment comprises a mobile console 112 and the patient area 120, whereby the mobile console 112 is operably associated with the patient area or patient bedside 120 at a physical distance. The mobile console 112 includes wheels or other mobile transport devices that allow the mobile console 112 to travel with the CPU 170. The mobile console 110 comprises the CPU 170 and a display 114 and the patient area 120 comprises the PIM engine 180 and the catheter 190. The PIM 180 includes the light source 130, the interferometer 140, the photodetector 150, and the digitizer 160. The light source 130 is operably associated with the interferometer 140 within the PIM 180, while the interferometer 140 operably associated with the sample probe 200 by the sample path 142. With the interferometer 140 included in the PIM 180, the sample path 142 does not traverse a substantial physical distance, but is rather locally connected with the catheter 190. The interferometer 140 is operably associated with the photodetector 150 in the PIM 180, while the photodetector 150 is operably associated with the digitizer 160 within the PIM 180. The digitizer 160 is operably associated with the CPU 170 in the mobile console 112 by way a PIM cable 162. The PIM cable 162 may be any connecting device and disconnected with the mobile console 112 through known connecting devices, female/male connectors, and the like. The CPU 170 in the control room 110 is for the imaging and processing of the images obtained from the catheter 190 and sample probe 200.

In another embodiment, the integrated OCT system 100 is shown in FIG. 2C, which is an extended digitizer-CPU laptop system. The integrated OCT system 100 in this embodiment comprises a laptop 116 and the patient area 120, whereby the laptop 116 is operably associated with the patient area or patient bedside 120 at a physical distance. The laptop 116 includes any computer-related device with a CPU 170, including, but not limited to netbooks, tablets, PDA's, mobile phones, music players, and the like, which may travel with the CPU 170. The laptop 116 comprises the CPU 170 and a display 114 and the patient area 120 comprises the PIM engine 180 and the catheter 190. The PIM 180 includes the light source 130, the interferometer 140, the photodetector 150, and the digitizer 160. The light source 130 is operably associated with the interferometer 140 within the PIM 180, while the interferometer 140 operably associated with the sample probe 200 by the sample path 142. With the interferometer 140 included in the PIM 180, the sample path 142 does not traverse a substantial physical distance, but is rather locally connected with the catheter 190. The interferometer 140 is operably associated with the photodetector 150 in the PIM 180, while the photodetector 150 is operably associated with the digitizer 160 within the PIM 180. The digitizer 160 is operably associated with the CPU 170 in the laptop 116 by way of PIM cable 162. The PIM cable 162 may be disconnected with the laptop 116 through known connecting devices, female/male connectors, USB connectors, video cables, HDMI cables, and the like. The CPU 170 in the control room 110 is for the imaging and processing of the images obtained from the catheter 190 and sample probe 200.

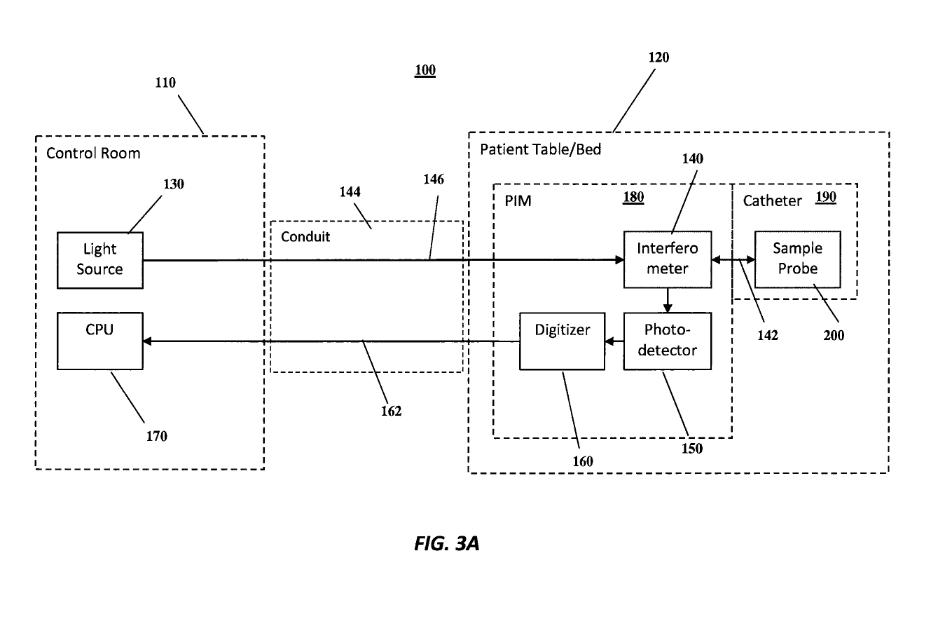

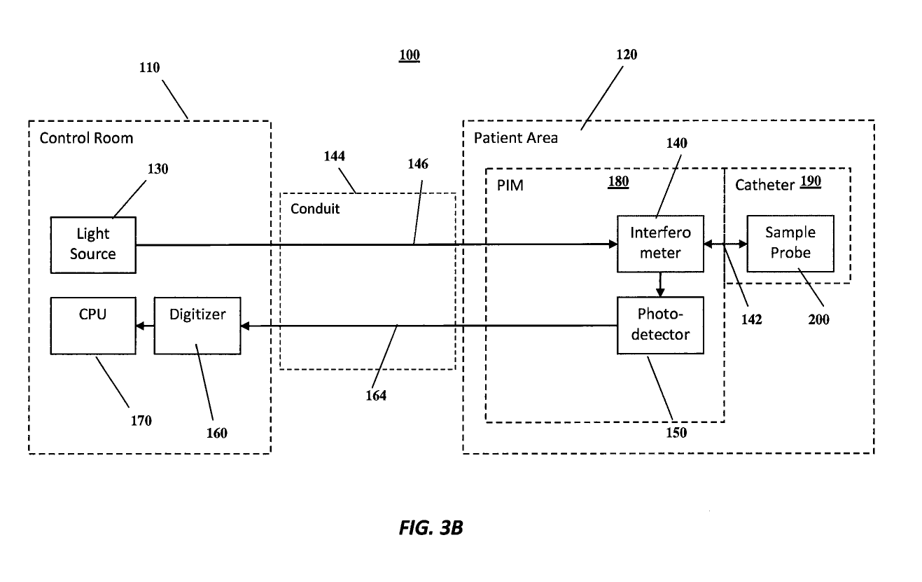

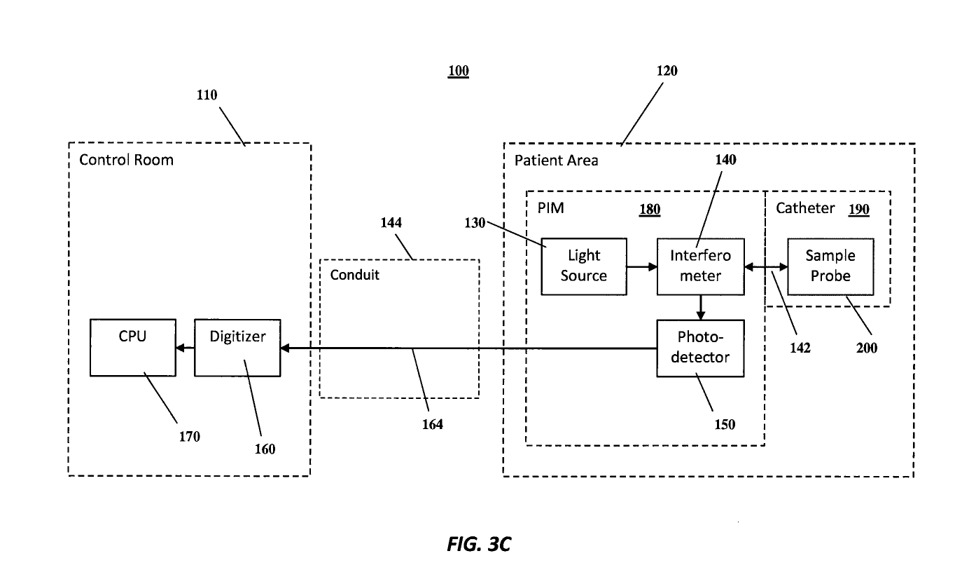

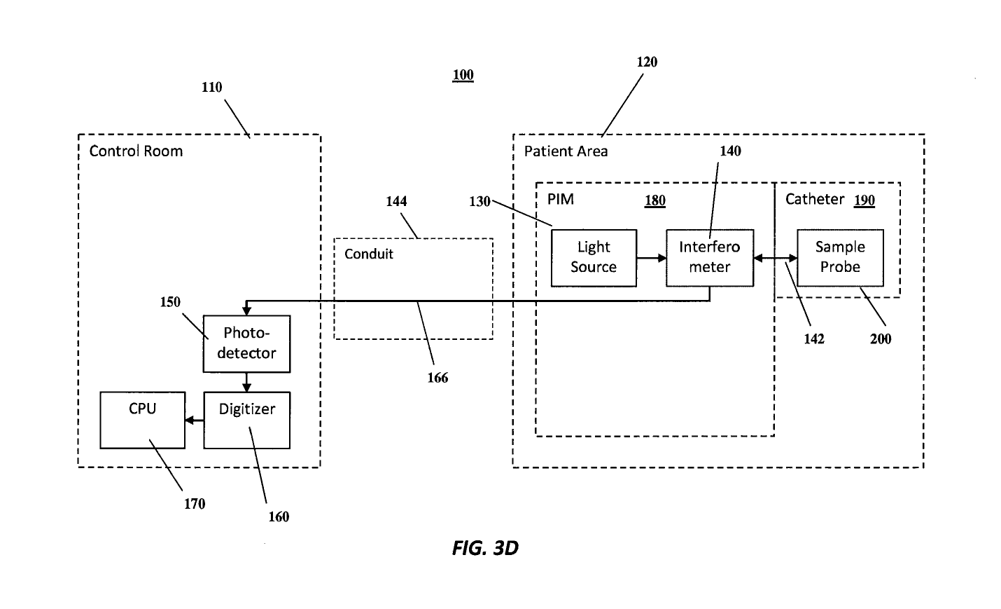

In another embodiment, the integrated OCT system 100 is shown in FIG. 3A, which is dual light path and PIM cable system. The integrated OCT system 100 in this embodiment comprises the control room 110 including the light source 130 and the CPU 170 while being operably associated with the Patient Table/Bed 120 that includes the PIM 180 and the catheter 190. The patient table 120 is located at a substantial physical distance from the control room 110. The light source 130 in the control room 110 is operably associated with the PIM 180 by way of a source path 146. The PIM 180 includes the interferometer 140, the photodetector 150, and the digitizer 160, whereby the interferometer 140 is operably associated with the source path 146. The interferometer 140 is further operably associated with the sample probe 200 by way of the sample path 142. With the interferometer 140 included in the PIM 180, the sample path 142 does not traverse a substantial physical distance, but is rather locally connected with the catheter 190 and the sample probe 200. The interferometer 140 is operably associated with the photodetector 150 in the PIM 180, while the photodetector 150 is operably associated with the digitizer 160 within the PIM 180. The digitizer 160 is operably associated with the CPU 170 in the control room 110 by way of PIM cable 162 through the conduit 144. The PIM cable 162 may be disconnected with the control room 110 through known connecting devices, female/male connectors, USB connectors, video cables, HDMI cables, and the like. The integrated OCT system 100 locates the OCT sample a substantial physical distance away from the OCT system's central processing/display/archival unit. The CPU in the control room 110 is for the imaging and processing of the images obtained from the catheter 190 and sample probe 200.

In another embodiment, the integrated OCT system 100 is shown in FIG. 3B, which is dual light path and PIM cable system. The integrated OCT system 100 in this embodiment comprises the control room 110 including the light source 130, the CPU 170, and the digitizer 160 while being operably associated with the Patient Table/Bed 120 that includes the PIM 180 and the catheter 190. The patient area 120 is located at a substantial physical distance from the control room 110. The light source 130 in the control room 110 is operably associated with the PIM 180 by way of a source path 146. The PIM 180 includes the interferometer 140 and the photodetector 150, whereby the interferometer 140 is operably associated with the source path 146. The interferometer 140 is further operably associated with the sample probe 200 by way of the sample path 142. With the interferometer 140 included in the PIM 180, the sample path 142 does not traverse a substantial physical distance, but is rather locally connected with the catheter 190 and the sample probe 200. The interferometer 140 is operably associated with the photodetector 150 in the PIM 180, while the photodetector 150 is operably associated with the digitizer 160 by way of a digitizer path 164 through the conduit 144. The digitizer 160 is operably associated with the CPU 170 in the control room 110. The digitizer path 164 may be disconnected with the control room 110 through known connecting devices, female/male connectors, USB connectors, video cables, HDMI cables, and the like. The CPU 170 in the control room 110 is for the imaging and processing of the images obtained from the catheter 190 and sample probe 200.

In another embodiment, the integrated OCT system 100 is shown in FIG. 3C, which is PIM integrated interferometer system. The integrated OCT system 100 in this embodiment comprises the control room 110 including the CPU 170 and the digitizer 160 while being operably associated with the patient area 120 that includes the PIM 180 and the catheter 190. The patient area 120 is located at a substantial physical distance from the control room 110. The PIM includes the light source 130, the interferometer 140, and the photodetector 150, whereby the interferometer 140 is operably associated with the photodetector 150 with the PIM 180. The interferometer 140 is further operably associated with the sample probe 200 by way of the sample path 142. With the interferometer 140 included in the PIM 180, the sample path 142 does not traverse a substantial physical distance, but is rather locally connected with the catheter 190 and the sample probe 200. The interferometer 140 is operably associated with the photodetector 150 in the PIM 180, while the photodetector 150 is operably associated with the digitizer 160 by way of a digitizer path 164 through the conduit 144. The digitizer 160 is operably associated with the CPU 170 in the control room 110. The digitizer path 164 may be disconnected with the control room 110 through known connecting devices, female/male connectors, USB connectors, video cables, HDMI cables, and the like. The CPU in the control room 110 is for the imaging and processing of the images obtained from the catheter 190 and sample probe 200.

In another embodiment, the integrated OCT system 100 is shown in FIG. 3D, which is PIM integrated interferometer system. The integrated OCT system 100 in this embodiment comprises the control room 110 including the CPU 170, the digitizer 160, and the photodetector 150 while being operably associated with the Patient Table/Bed 120 that includes the PIM 180 and the catheter 190. The patient area 120 is located at a substantial physical distance from the control room 110. The PIM includes the light source 130 and the interferometer 140, whereby the interferometer 140 is operably associated with the photodetector 150 by way of a detection path 166. The interferometer 140 is further operably associated with the sample probe 200 by way of the sample path 142. With the interferometer 140 included in the PIM 180, the sample path 142 does not traverse a substantial physical distance, but is rather locally connected with the catheter 190 and the sample probe 200. The interferometer 140 is operably associated with the photodetector 150 by way of the detection path 166 through the conduit 144. The photodetector 150 in the control room 110 is operably associated with the digitizer 160 and the digitizer 160 is operably associated with the CPU 170 in the control room 110. The detection path 166 may be disconnected with the control room 110 through known connecting devices, female/male connectors, USB connectors, video cables, HDMI cables, and the like. The CPU in the control room 110 is for the imaging and processing of the images obtained from the catheter 190 and sample probe 200.

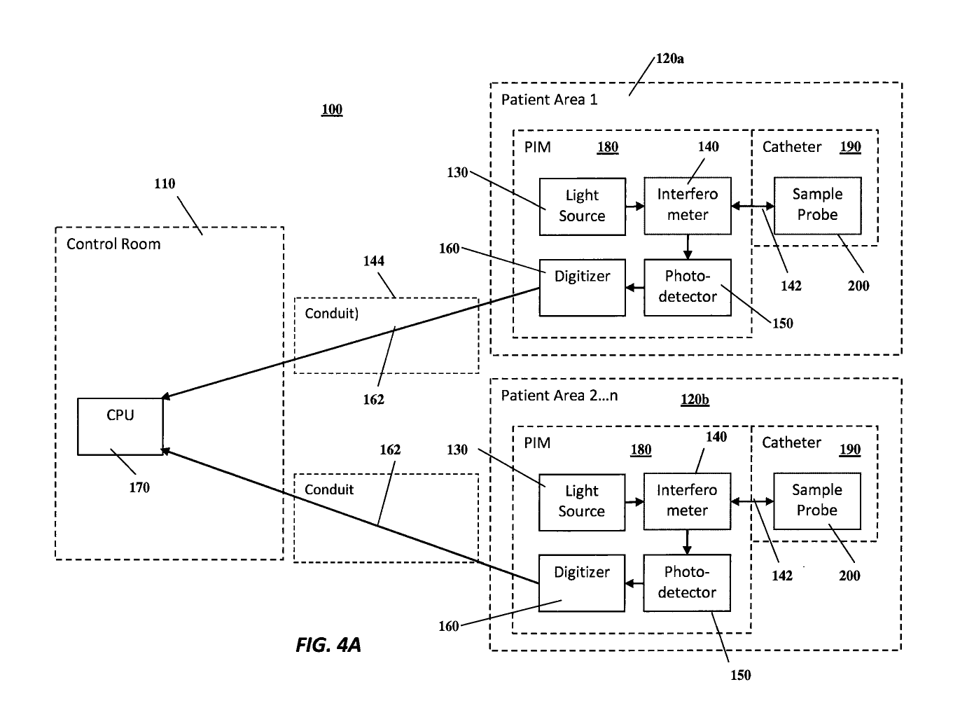

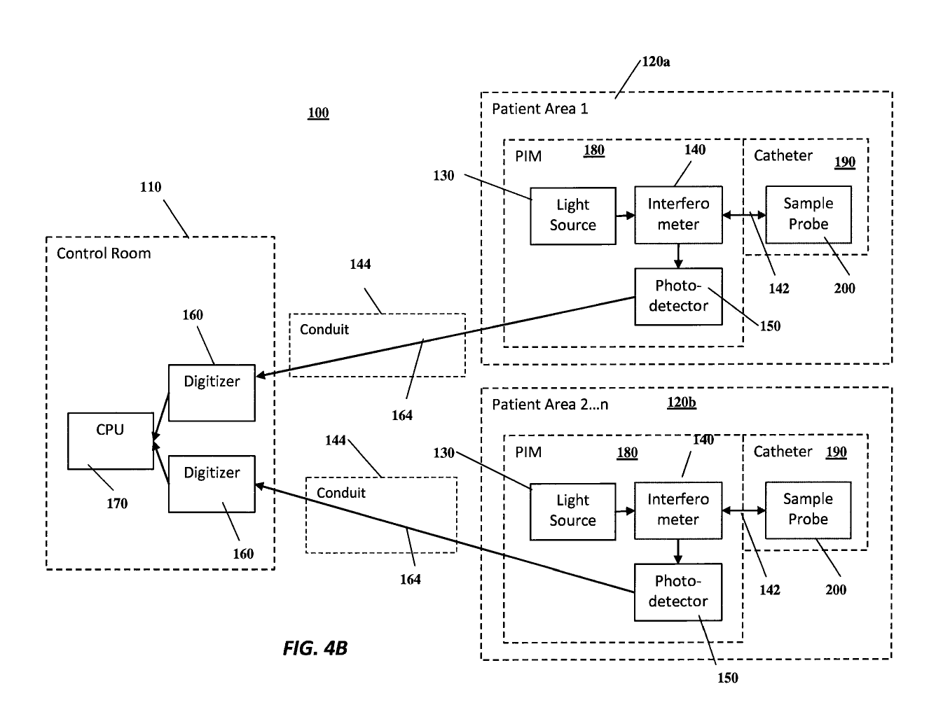

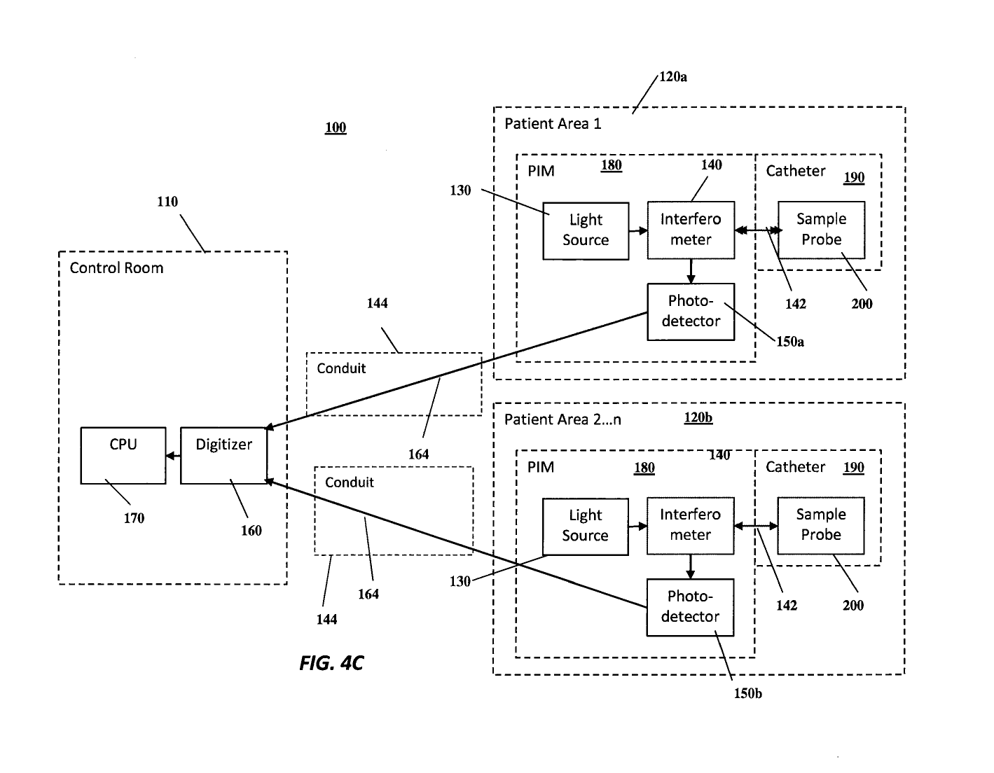

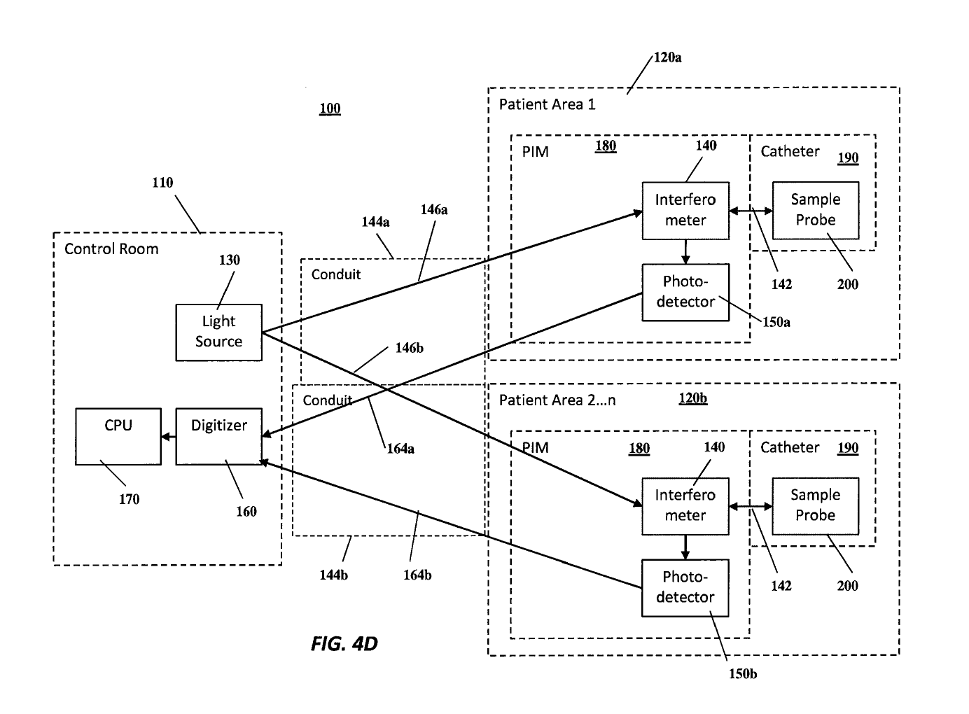

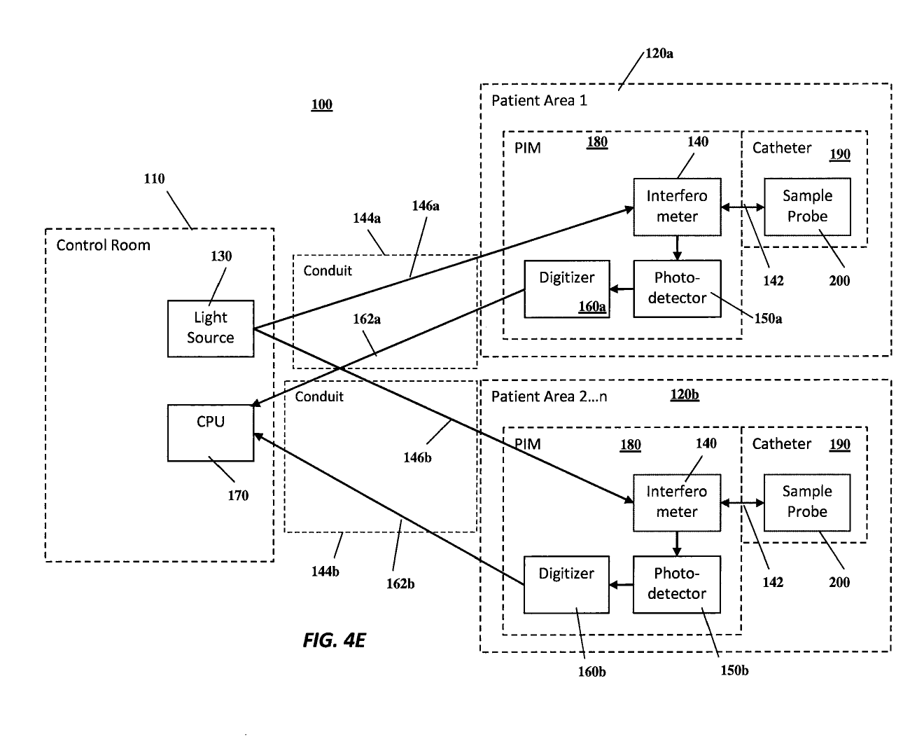

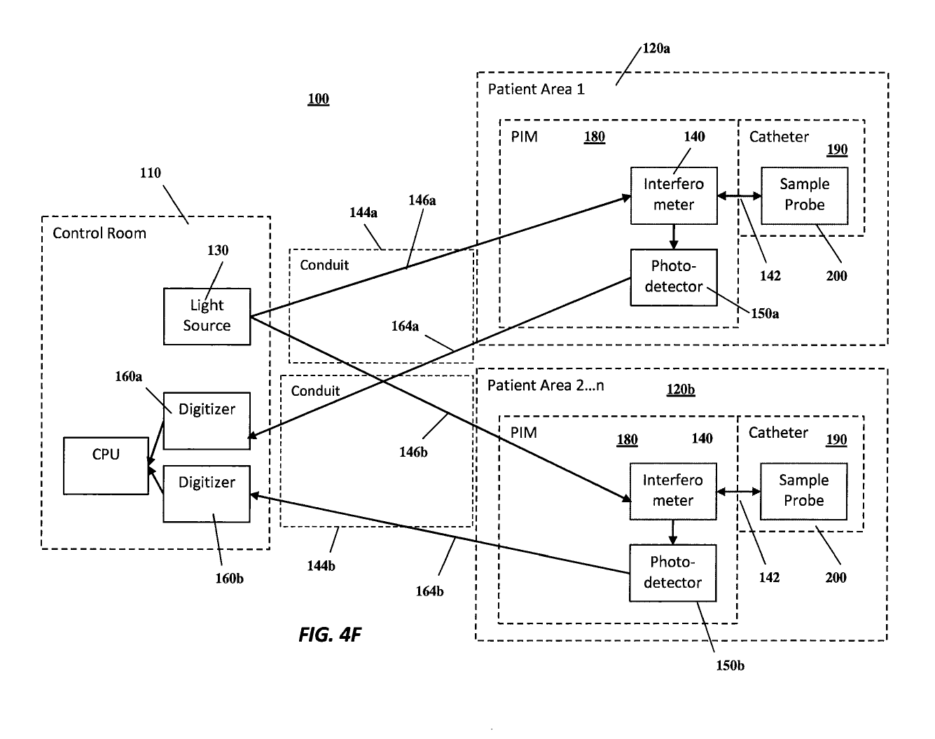

In another embodiment, the integrated OCT system 100 is shown in FIG. 4A, which is a distributed interferometer system. The integrated OCT system 100 in this embodiment comprises the control room 110 including the CPU 170 operably associated with at least two patient areas 120a and 120b. The patient areas 120a and 120b effectively distribute the CPU 170 capabilities to multiple patient areas when the control room 110 is located at a substantial physical distance away from such patient areas 120a and 120b. The patient areas 120a and 120b include the PIM 180 and the catheter 190, whereby the PIM 180 includes the light source 130, the interferometer 140, the photodetector 150, and the digitizer 160. The interferometer 140 is operably associated with the sample probe 200 in the catheter 190 by the sample path 142. The digitizer 160 in the PIM 180 is operably associated with the CPU 170 in the control room 110 by way of the CPU path 162. The CPU 170 is operable with multiple inputs for the CPU paths 162, as to accept multiple CPU paths 162 from multiple PIMs 180 and patient areas 120a and 120b. The integrated OCT system 100 locates the OCT sample a substantial physical distance away from the OCT system's central processing/display/archival unit. The CPU 170 in the control room 110 is for the imaging and processing of the images obtained from the catheter 190 and sample probe 200.

In another embodiment, the integrated OCT system 100 is shown in FIG. 4B, which is another distributed interferometer system. The integrated OCT system 100 in this embodiment comprises the control room 110 including the CPU 170 and at least two digitizers 160a and 160b operably associated with at least two patient areas 120a and 120b, respectively. The patient areas 120a and 120b and the two digitizers 160a and 160b effectively distribute the CPU 170 capabilities to multiple patient areas when the control room 110 is located at a substantial physical distance away from such patient areas 120a and 120b. The patient areas 120a and 120b include the PIM 180 and the catheter 190, whereby the PIM 180 includes the light source 130, the interferometer 140, and the photodetector 150. The interferometer 140 is operably associated with the sample probe 200 in the catheter 190 by the sample path 142. The photodetector 150 is operably associated with the digitizers 160a and 160b in the control room 110 by way of the digitizer path 164. The CPU 170 is operable with multiple inputs for the digitizers 160a and 160b, as to accept multiple photodetectors 150 from multiple PIMs 180 and patient areas 120a and 120b. The CPU 170 in the control room 110 is for the imaging and processing of the images obtained from the catheter 190 and sample probe 200.

In another embodiment, the integrated OCT system 100 is shown in FIG. 4C, which is another distributed interferometer system. The integrated OCT system 100 in this embodiment comprises the control room 110 including the CPU 170 and a single digitizer 160 operably associated with at least two patient areas 120a and 120b. The patient areas 120a and 120b and the digitizers 160 effectively distribute the CPU 170 capabilities to multiple patient areas when the control room 110 is located at a substantial physical distance away from such patient areas 120a and 120b. The patient areas 120a and 120b include the PIM 180 and the catheter 190, whereby the PIM 180 includes the light source 130, the interferometer 140, and the photodetector 150. The interferometer 140 is operably associated with the sample probe 200 in the catheter 190 by the sample path 142. The photodetectors 150a and 150b are operably associated with the digitizer 160 in the control room 110 by way of the digitizer paths 164. The digitizer 160 is operable with multiple inputs for the digitizer paths 164, as to accept multiple photodetectors 150 from multiple PIMs 180 and patient areas 120a and 120b. The CPU 170 in the control room 110 is for the imaging and processing of the images obtained from the catheter 190 and sample probe 200.

In another embodiment, the integrated OCT system 100 is shown in FIG. 4D, which is another distributed interferometer system. The integrated OCT system 100 in this embodiment comprises the control room 110 including the light source 130, the digitizer 160, and the CPU 170, whereby the light source 130 is operably associated with at least two patient areas 120a and 120b. The patient areas 120a and 120b effectively distribute the light source's 130 capabilities to multiple patient areas when the control room 110 is located at a substantial physical distance away from such patient areas 120a and 120b. The patient areas 120a and 120b include the PIM 180 and the catheter 190, whereby the PIM 180 includes the interferometer 140 and the photodetector 150. The light source 130 is operably associated with the interferometers 140 in the PIMs 180 by the light paths 146a and 146b through the conduits 144a and 144b. The interferometers 140 are operably associated with the sample probe 200 in the catheter 190 by the sample path 142. The photodetectors 150a and 150b are operably associated with the digitizer 160 in the control room 110 by way of the digitizer paths 164a and 164b. The digitizer 160 is operable with multiple inputs for the digitizer paths 164a and 164b, as to accept multiple photodetectors 150 from multiple PIMs 180 and patient areas 120a and 120b. The digitizer 160 is operably associated with the CPU 170 in the control room 110 for imaging and processing.

In another embodiment, the integrated OCT system 100 is shown in FIG. 4E, which is another distributed interferometer system. The integrated OCT system 100 in this embodiment comprises the control room 110 including the light source 130 and the CPU 170, whereby the light source 130 is operably associated with at least two patient areas 120a and 120b. The patient areas 120a and 120b effectively distribute the light source's 130 capabilities to multiple patient areas when the control room 110 is located at a substantial physical distance away from such patient areas 120a and 120b. The patient areas 120a and 120b include the PIM 180 and the catheter 190, whereby the PIM 180 includes the interferometer 140, the photodetector 150, and the digitizers 160a and 160b. The light source 130 is operably associated with the interferometers 140 in the PIMs 180 by the light paths 146a and 146b through the conduits 144a and 144b. The interferometers 140 are operably associated with the sample probe 200 in the catheter 190 by the sample path 142. The photodetectors 150a and 150b are operably associated with the digitizers 160a and 160b in the PIM 180. The digitizers 160a and 160b are operable with the CPU 170 in the control room 110 by CPU paths 162a and 162b. The CPU 170 includes multiple inputs for the CPU paths 162a and 162b, as to accept multiple digitizers 160a and 160b from multiple PIMs 180 and patient areas 120a and 120b. The CPU 170 in the control room 110 is for the imaging and processing of the images obtained from the catheter 190 and sample probe 200.

In another embodiment, the integrated OCT system 100 is shown in FIG. 4F, which is another distributed interferometer system. The integrated OCT system 100 in this embodiment comprises the control room 110 including the light source 130, the digitizers 160a and 160b, and the CPU 170, whereby the light source 130 is operably associated with at least two patient areas 120a and 120b. The patient areas 120a and 120b effectively distribute the light source's 130 capabilities to multiple patient areas when the control room 110 is located at a substantial physical distance away from such patient areas 120a and 120b. The patient areas 120a and 120b include the PIM 180 and the catheter 190, whereby the PIM 180 includes the interferometer 140 and the photodetectors 150a and 150b. The light source 130 is operably associated with the interferometers 140 in the PIMs 180 by the light paths 146a and 146b through the conduits 144a and 144b. The interferometers 140 are operably associated with the sample probe 200 in the catheter 190 by the sample path 142. The photodetectors 150a and 150b are operably associated with the interferometer 140 and with the digitizers 160a and 160b in the control room 110 by way of the digitizer paths 164a and 164b from multiple PIMs 180 and patient areas 120a and 120b. The digitizers 160a and 160b are operably associated with the CPU 170 in the control room 110, such that the CPU 170 is able to accept multiple digitizers 160a and 160b. The CPU 170 in the control room 110 processes the images from multiple patient areas.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. An integrated system comprising:
a work station comprising a imaging optics; and
a patient area comprising a sampling probe;
wherein the work station is remote from the patient area and the work station is operably associated with the patient area.

2. The system according to claim 1, wherein the imaging optics are for an imaging system selected from a group consisting of an Optical Coherence Tomography (OCT) system; a spectroscopic device including fluorescence, absorption, scattering, and Raman spectroscopies, an intravascular ultrasound (IVUS) device, a Forward-Looking IVUS (FLIVUS) device, a high intensity focused ultrasound (HIFU) device, a radiofrequency device, a thermal imaging device, an optical light-based imaging device, a magnetic resonance device, a radiography device, a nuclear imaging device, a photoacoustic imaging device, an electrical impedance tomography device, an elastography device, a pressure sensing wire device, an intracardiac echocardiography (ICE) device, a forward looking ICE device, an orthopedic device, a spinal imaging device, a neurological imaging device, an image guided therapeutic device, a therapeutic delivery device, and a diagnostic delivery device.

3. The system according to claim 1, wherein the work station further comprises a photodetector, a digitizer, and a CPU component.

4. The system according to claim 1, wherein the patient area further comprises a catheter.

5. The system according to claim 1, wherein the interferometer comprises an extended sample path operably associated with the sample probe.

6. The integrated system of claim 1, wherein the remote work station is operably associated with the patient area by at least one cable.

7. The integrated system of claim 1, wherein the at least one cable is installed through a floor trench or a ceiling conduit.

8. The integrated system of claim 1, wherein the remote work station is operably associated with the patient area by wireless transmission.

9. An integrated optical coherence tomography (OCT) system comprising:
a work station comprising a light source and an interferometer; and
a patient area comprising a sample probe;
wherein the work station is remote from the patient area and the work station is operably associated with the patient area.

10. The system according to claim 9, wherein the work station further comprises a photodetector, a digitizer, and a CPU component.

11. The system according to claim 9, wherein the patient area further comprises a catheter.

12. The system according to claim 9, wherein the interferometer comprises an extended sample path operably associated with the sample probe.

13. The integrated system of claim 9, wherein the remote work station is operably associated with the patient area by at least one cable.

14. The integrated system of claim 9, wherein the at least one cable is installed through a floor trench or a ceiling conduit.

15. The integrated system of claim 9, wherein the remote work station is operably associated with the patient area by wireless transmission.

16. A method of integrating systems, the method comprising:
providing a work station comprising a imaging optics;
providing a patient area comprising a sampling probe;
separating the work station from the patient area such that the work station is remote from the patient area while still being operably associated with the patient area; and
sending image data from the patient area to the work station.

17. The method of claim 16, further comprising converting the image data to digital form at or near the work station.

18. The method of claim 16, further comprising converting the image data to digital form before the image data is sent from the patient area.

19. The method of claim 16, further comprising compressing the image data before sending the image data from the patient area.

20. The method of claim 16, wherein the imaging optics are part of an imaging system that is selected from a group consisting of an Optical Coherence Tomography (OCT) system; a spectroscopic device including fluorescence, absorption, scattering, and Raman spectroscopies, an intravascular ultrasound (IVUS) device, a Forward-Looking IVUS (FLIVUS) device, a high intensity focused ultrasound (HIFU) device, a radiofrequency device, a thermal imaging device, an optical light-based imaging device, a magnetic resonance device, a radiography device, a nuclear imaging device, a photoacoustic imaging device, an electrical impedance tomography device, an elastography device, a pressure sensing wire device, an intracardiac echocardiography (ICE) device, a forward looking ICE device, an orthopedic device, a spinal imaging device, a neurological imaging device, an image guided therapeutic device, a therapeutic delivery device, and a diagnostic delivery device.
